(19) United States
(12) Patent Application Publication
(10) Pub. No.: US 2015/0133310 A1
(43) Pub. Date: May 14, 2015

(54) NUCLEIC ACID SEQUENCING SYSTEMS AND METHODS

(71) Applicant: IBIS BIOSCIENCES, INC., Carlsbad, CA (US)

(72) Inventor: Mark A. Hayden, Ingleside, IL (US)

(21) Appl. No.: 14/398,263

(22) PCT Filed: May 2, 2013
(86) PCT No.: PCT/US13/39294

§ 371 Date: Oct. 31, 2014

Related U.S. Application Data
(60) Provisional application No. 61/641,731, filed on May 2, 2012.

(51) Int. Cl. C12Q 1/68; C12N 15/10

(52) U.S. Cl. CPC C12Q 1/6874; C12N 15/1065
USPC 506/2; 506/26; 506/16

(57) ABSTRACT

The present invention relates to systems and methods for performing isothermal amplification reactions. In particular, the present invention relates to denaturation methods for use in isothermal amplification reactions.

### CROSS-REFERENCE TO RELATED APPLICATIONS

The present Application claims priority to U.S. Provisional Application Ser. No. 61/641,731 filed 2 May 2012, the entirety of which is incorporated by reference herein.

### FIELD OF INVENTION

The present invention relates to systems and methods for performing nucleic acid sequencing reactions. In particular, the present invention relates to pre-amplification systems and methods that improve the efficiency for subsequent sequencing reactions.

### BACKGROUND

Nucleic acid sequencing technologies that employ highly massive parallel sequencing-by-synthesis (SBS) strategies have extremely high throughput that has allowed for inexpensive whole genome sequencing. There are, however, several limitations to the upfront workflow process that can result in long processing times with many steps that are difficult to automate. One such step involves the enrichment of microparticle beads containing clonal amplicons by separating from beads without amplified material after emulsion PCR. This allows for upfront enrichment that improves the subsequent sequencing reaction by allowing for a higher percentage of picoliter reaction wells to be filled with target amplicon, as opposed to empty beads without amplicon. Another limitation surrounds technologies that directly amplify nucleic acid target on a solid surface using solid-phase PCR. Traditionally, solid phase PCR is much less efficient than solution based PCR, which results in lower quantities of amplicon being amplified. This lower level of amplicon reduces the detection signal in the subsequent SBS sequencing reaction. This can add a significant amount of time to the detection step because it takes longer to detect a signal above background when lower amounts of template are present.

Methods for improving sensitivity and robustness of sequencing assays are needed.

### SUMMARY

The present invention relates to systems and methods for performing nucleic acid sequencing reactions. In particular, the present invention relates to pre-amplification systems and methods that improve the efficiency for subsequent sequencing reactions.

For example, in some embodiments, the present invention provides a method, comprising: a) amplifying a plurality of nucleic acid segments with a reaction mixture comprising a template and first and second primers, at least one of said first and second primers covalently attached to an agarose droplet to generate double stranded amplified target covalently attached to the agarose droplet; b) denaturing the double stranded amplified target to generate single stranded amplified target attached to the agarose droplet; and c) purifying agarose droplets comprising the single stranded amplified target from agarose droplets not comprising the single stranded amplified target. In some embodiments, the method further comprises the step of sequencing the single stranded amplified target (e.g., next generation sequencing or sequencing by synthesis, optionally automated and/or performed on a solid support). In some embodiments, the denaturing comprises the use of a chemical denaturant (e.g., sodium hydroxide). In some embodiments, the denaturing further comprises a wash step. In some embodiments, the purifying step comprises flow cytometry. In some embodiments, the flow cytometry utilizes a fluorescent probe or fluorescent intercalator dye. In some embodiments, the agarose beads are between 1 nanoliter and 1 picoliter in size. In some embodiments, the agarose beads comprise a chemical moiety (e.g., biotin or an antibody) for imbolization onto a solid surface (e.g., a sequencing solid support or surface).

Additional embodiments provide kits and systems comprising reagents necessary, sufficient or useful for performing the aforementioned amplification and sequencing analysis.

Additional embodiments will be apparent to persons skilled in the relevant art based on the teachings contained herein.

### DESCRIPTION OF THE FIGURES

FIG. 1 shows an overview of exemplary agarose droplets used in some embodiments of the present invention.

FIG. 2 shows exemplary denaturation methods.

FIG. 3 shows exemplary methods for purification of agarose beads with amplified target nucleic acid sequences.

FIG. 4 shows exemplary methods of attaching agarose beads to solid supports.

### DETAILED DESCRIPTION

The present invention relates to systems and methods for performing multiplex amplification reactions. In particular, the present invention relates to one-reaction multiplex pre-amplification systems and methods.

### Definitions

To facilitate an understanding of the present technology, a number of terms and phrases are defined below. Additional definitions are set forth throughout the detailed description.

As used herein, “a” or “an” or “the” can mean one or more than one. For example, “a” widget can mean one widget or a plurality of widgets.

The term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer should be sufficiently long to prime the synthesis of extension products in the presence of the inducing agent. The exact lengths of the primers will depend on many factors, including temperature, source of primer and the use of the method.

The term “target,” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence.

“Solid support” as used herein refers to any solid surface to which nucleic acids can be attached, such as for example, including but not limited to, metal surfaces, latex beads, dextran beads, polystyrene surfaces, polypropylene surfaces, polyacrylamide gel, gold surfaces, glass surfaces and silicon wafers.

“Means for immobilizing nucleic acids to a solid support” as used herein refers to any chemical or non-chemical attachment method including chemically-modifiable functional groups. “Attachment” relates to immobilization of nucleic acid on solid supports by either a covalent attachment or a non-covalent attachment.

As used herein, the terms “subject” and “patient” refer to any animal, such as a dog, a cat, a bird, livestock, and particularly a mammal, and preferably a human.

As used herein, the term “sample” is used in its broadest sense. In one sense, it is meant to include a representative portion or culture obtained from any source, including biological and environmental sources. Biological samples may be obtained from animals (including humans) and encompass fluids, solids, tissues, and gases. Biological samples include blood products, such as plasma, serum, and the like. Environmental samples include environmental material such as surface matter, soil, mud, sludge, biofilms, water, and industrial samples. Such examples are not however to be construed as limiting the sample types applicable to the present invention.

### Embodiments of the Technology

Although the disclosure herein refers to certain illustrated embodiments, it is to be understood that these embodiments are presented by way of example and not by way of limitation.

Embodiments of the present invention provide compositions and methods for improving PCR efficiency (e.g., for subsequence sequencing reactions). In some embodiments, systems and methods use solid support (e.g., agarose-based digital) PCR to clonally amplify nucleic acid. Such digital agarose beads can be easily sorted into reactive or nonreactive beads by flow sorting, which is highly automatable. Thus, in some embodiments, an enriched population of amplicon-positive beads is used to fill up a downstream sequencing reaction vessel (e.g., a reaction vessel containing picoliter reaction wells or a flat surface flowcell format). The net result is an increase in PCR efficiency and detection signal, in addition to a faster and more efficient workflow with less hands-on time.

In some embodiments, agarose droplet emulsion PCR (Leng et al., Lab Chip 10:2841 (2010; Yang et al., “14^{th} International Conference on Miniaturized Systems for Chemistry and Life Sciences, Oct. 3-7, 2010, Groningen, The Netherlands; each of which is herein incorporated by reference) is utilized to amplify sequencing targets. In some embodiments, the amplification products are directly fed into next generation sequencing systems for efficient and fast sequencing reactions (e.g., using automated methods).

Droplet PCR (e.g., agarose droplet PCR) capitalizes on the unique thermoresponsive sol-gel switching property of agarose for highly efficient DNA amplification and amplicon trapping. Uniform agarose solution droplets generated via a microfluidic chip serve as robust and inert nanolitre PCR reactors for amplification. After PCR, agarose droplets are gelated to form agarose beads, trapping all amplicons in each reactor. This method does not require cocapsulation of primer labeled microbeads, allows high throughput generation of uniform droplets and enables high PCR efficiency, making it a useful platform for many downstream applications.

Several known next generation sequencing chemistries (See e.g., below discussion of sequencing methods) utilize amplification on a solid surface, which is inefficient. One possible solution would be to run standard solution-based PCR, which is highly efficient, and use the amplified material for subsequent SBS reaction chemistries. However, this strategy is not conducive to SBS since the amplicon is not immobilized on a solid surface. Without an immobilized amplicon, the material is washed away during the SBS sequencing chemistry steps.

In some embodiments, agarose-based digital PCR is used to circumvent this limitation. FIG. 1 illustrates an exemplary method of generating agarose droplets. For example, in some embodiments, PCR primers are covalently attached to agarose via Schiff-base reaction and made into aqueous gel droplets by using microfluidics to create droplets in carrier oil (Leng et al., supra). Droplets of different sizes are made, depending on the application and reaction vessel. These droplets form standard digital droplets in that they contain agarose, which normally melts above 50° C., depending on the concentration. During the PCR reaction, the agarose droplets melt and PCR occurs with similar efficiency as standard PCR. In some embodiments, one or both of the primers is immobilized on the agarose, resulting in the double-stranded PCR amplicon being physically attached to the gel matrix at the conclusion of the PCR reaction.

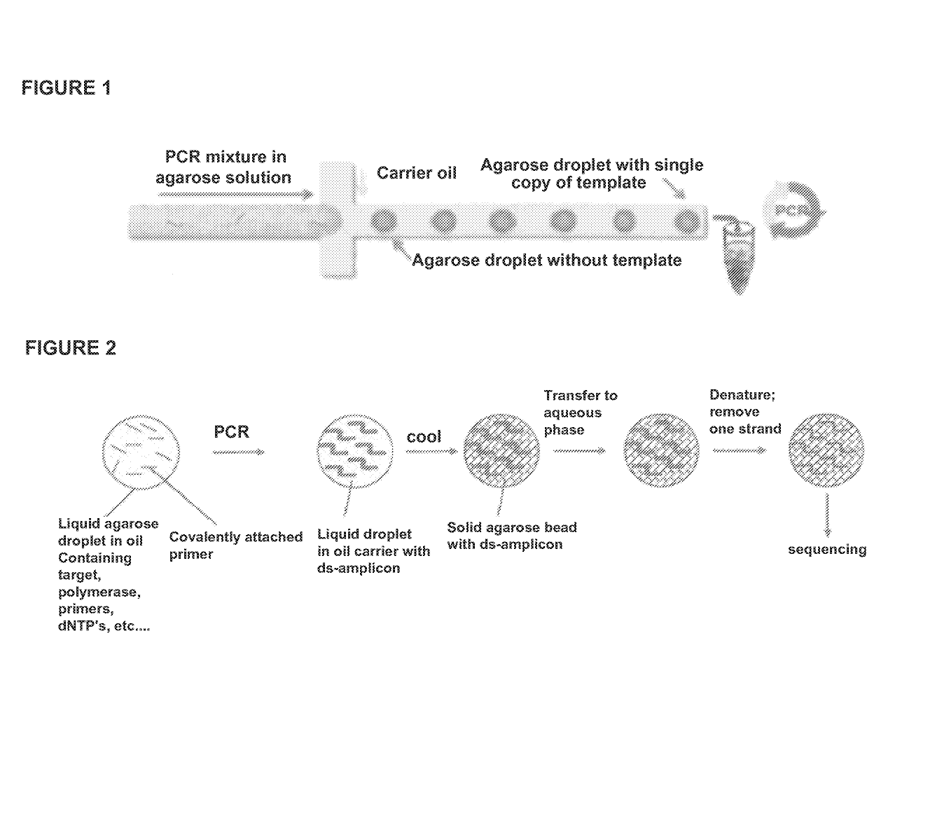

In some embodiments, the next step in the process converts the double-stranded amplicon to single-stranded target for sequencing (FIG. 2). Since one of the amplicon strands is covalently attached to the agarose matrix, the other strand is easily denatured (e.g., by incubating the beads in an aqueous solution containing a denaturant (e.g., NaOH)). The small size of the agarose beads (nanoliter to picoliter) allows fast diffusion of chemical denaturants in and out of the gel matrix, including the released amplicon strand. In some embodiments, wash steps are included to efficiently remove any released amplicon or other reagents. The resulting beads each contain a clonal population of single stranded amplicon ready for sequencing.

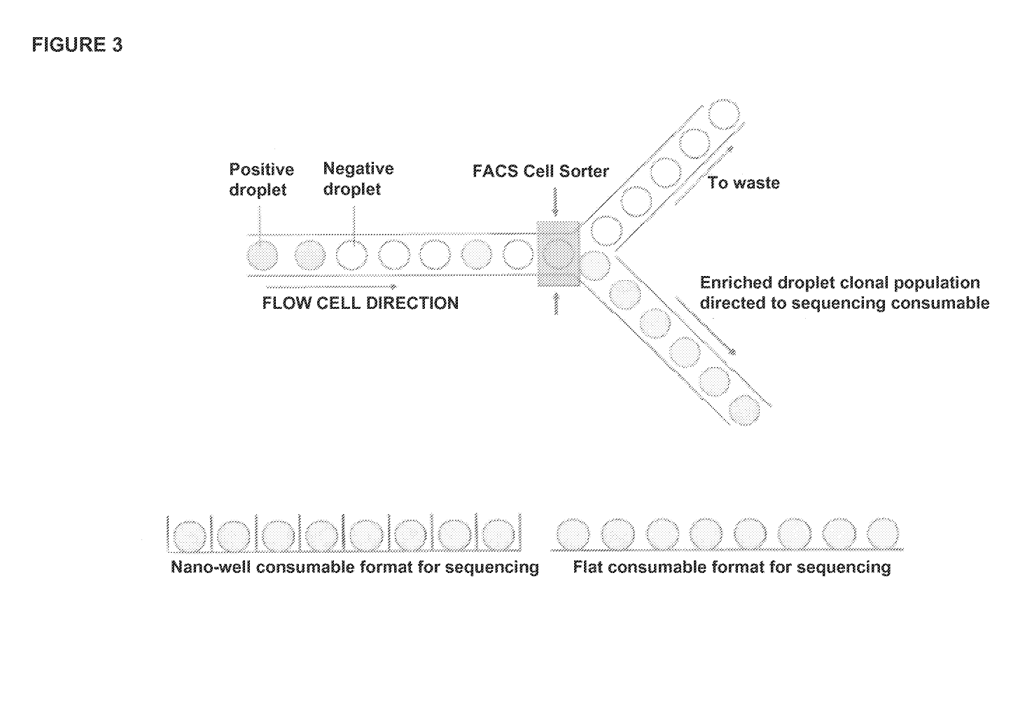

In some embodiments, a method of enriching for droplets containing amplicon is included. In some embodiments, fluorescence is used as a marker for identification of amplification products. For example, in some embodiments, sorting by flow cytometry where a fluorescent probe or nonspecific intercalator dye (e.g., SYBR Green. ethidium bromide, etc) is used to identify droplets with or without amplicon, although other separation and enrichment methods may be utilized. These are then sorted into different bin locations using fluorescence-activated cell sorting (FACS) technology. This provides a fast and efficient way to enrich for positive droplets over ones that contain no amplicon. The separation and enrichment systems and methods described herein are highly automatable and rapid, requiring very little hands-on time, in sharp contrast to enrichment methods utilized in protocols from other NGS methods, where numerous manual steps and multiple instrument systems are required to enrich microparticles. Once an enriched population of beads is selected, they can be deposited on the surface of an appropriate consumable for subsequent sequencing (FIG. 3).

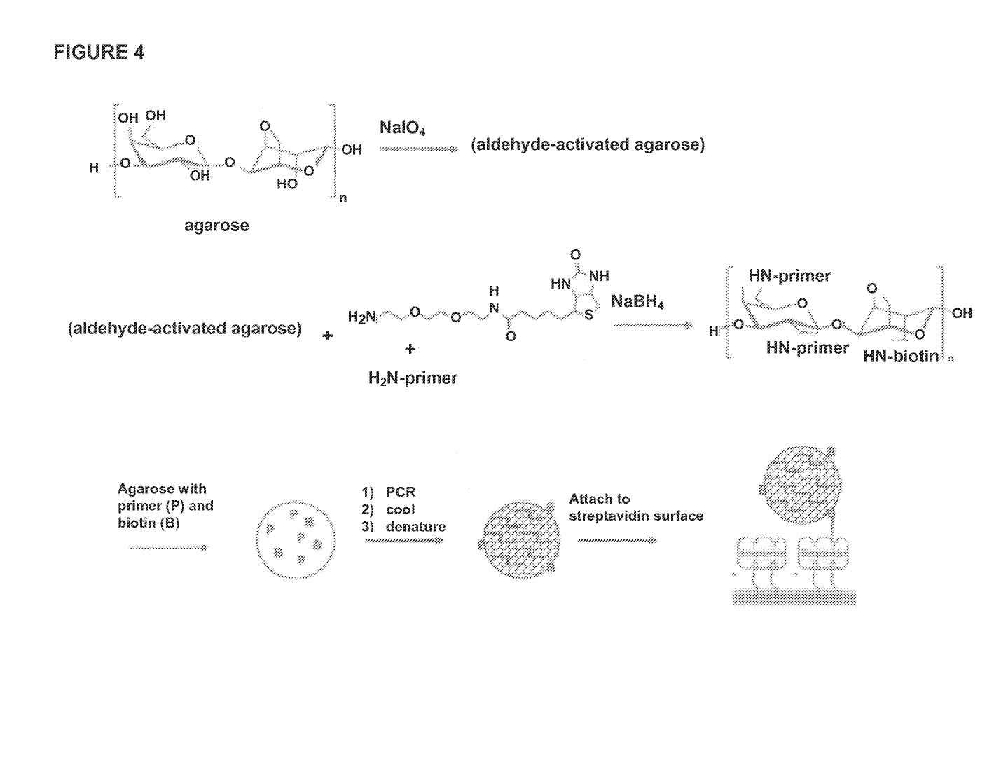

In some embodiments, an appropriate bead size is selected for depositing the solid agarose beads into nanowells for sequencing. In some embodiments, beads are sized to fit in each well while excluding other beads. Alternatively, the agarose is derivatized with a suitable chemical moiety for immobilization on a solid surface. There are numerous immobilization chemistry options available for attaching the agarose beads to a surface. For example, in some embodiments, the coupling of biotin to the agarose matrix during the step where the primer is covalently attached to the gel matrix is utilized. In this example, a 5′ amino-linked primer and amino-linked biotin are chemically coupled to agarose using sodium metaperiodate (NaIO₄) oxidation of the agarose hydroxyl groups to form aldehyde groups, which forms a Schiff base when coupled to amine groups (FIG. 4). Reduction by sodium borohydride (NaBH₄) gives a stable secondary amine. Amine-labeled biotin is commercially available for such applications and sold under the trade name Amine-PEG-Biotin. Alternatively, other coupling strategies are used such as antibody-hapten or ionic interactions, or other schemes. Once the beads are on the surface, they are subjected to standard sequencing chemistry, such as SBS or similar chemistry, to determine the nucleic acid sequence.

The systems and methods described herein find use in a variety of sequencing applications. Exemplary sequencing methods are described below.

In some embodiments, the technology provided herein finds use in a Second Generation (a.k.a. Next Generation or Next-Gen), Third Generation (a.k.a. Next-Next-Gen), or Fourth Generation (a.k.a. N3-Gen) sequencing technology including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence. In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, the technology finds use in nanopore sequencing (see, e.g., Astier et al., J. Am. Chem. Soc. 2006 February 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, the technology finds use in HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

The technology finds use in another nucleic acid sequencing approach developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

In some embodiments, capillary electrophoresis (CE) is utilized to analyze amplification fragments. During capillary electrophoresis, nucleic acids (e.g., the products of a PCR reaction) are injected electrokinetically into capillaries filled with polymer. High voltage is applied so that the fluorescent DNA fragments are separated by size and are detected by a laser/camera system. In some embodiments, CE systems from Life Technogies (Grand Island, N.Y.) are utilized for fragment sizing (See e.g., U.S. Pat. No. 6,706,162, U.S. Pat. No. 8,043,493, each of which is herein incorporated by reference in its entirety).

In some embodiments, the present invention provides kits and systems for the amplification of nucleic acids for use in sequencing reactions. In some embodiments, kits include reagents necessary, sufficient or useful for amplification of nucleic acids (e.g., primers, agarose beads, solid supports, reagents, controls, instructions, etc.). In some embodiments, systems include automated sample and reagent handling devices (e.g., robotics).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method, comprising:
a) amplifying a plurality of nucleic acid segments with a reaction mixture comprising a template and first and second primers, at least one of said first and second primers covalently attached to an agarose droplet to generate double stranded amplified target covalently attached to said agarose droplet;
b) denaturing said double stranded amplified target to generate single stranded amplified target attached to said agarose droplet; and
c) purifying agarose droplets comprising said single stranded amplified target from agarose droplets not comprising said single stranded amplified target.

2. The method of claim 1, further comprising the step of sequencing said single stranded amplified target.

3. The method of claim 2, wherein said sequencing is sequencing by synthesis.

4. The method of claim 1, wherein said denaturing comprises the use of a chemical denaturant.

5. The method of claim 4, wherein said chemical denaturant is sodium hydroxide.

6. The method of claim 1, wherein said denaturing further comprises a wash step.

7. The method of claim 1, wherein said purifying comprises flow cytometry.

8. The method of claim 7, wherein said flow cytometry utilizes a fluorescent probe or fluorescent intercalator dye.

9. The method of claim 1, wherein said agarose beads are between 1 nanoliter and 1 picoliter in size.

10. The method of claim 1, wherein said agarose beads comprise a chemical moiety for imbolization onto a solid surface.

11. The method of claim 10, wherein said chemical moiety is biotin.

12. The method of claim 10, wherein said chemical moiety is an antibody.

13. A method, comprising:
a) amplifying a plurality of nucleic acid segments with a reaction mixture comprising a template and first and second primers, at least one of said first and second primers covalently attached to an agarose droplet to generate double stranded amplified target covalently attached to said agarose droplet;
b) denaturing said double stranded amplified target to generate single stranded amplified target attached to said agarose droplet;
c) purifying agarose droplets comprising said single stranded amplified target from agarose droplets not comprising said single stranded amplified target; and
d) sequencing said single stranded amplified target.

14. A system, comprising:
a) a plurality of first and second amplification primers, wherein at least one of said first and second primers covalently attached to an agarose droplet;
b) reagents for performing an amplification reaction with said primers;
c) reagents for denaturing double stranded amplified target; and
d) an apparatus for purifying agarose beads comprising single stranded amplified target.

15. The system of claim 14, further comprising an apparatus for sequencing said single stranded amplified target.

16. The system of claim 14, wherein said reagents for denaturing said double stranded amplified target is a chemical denaturant.

17. The system of claim 15, wherein said chemical denaturant is sodium hydroxide.

18. The system of claim 14, wherein said apparatus for purifying is a flow cytometry apparatus.

19. The system of claim 14, wherein said agarose beads are between 1 nanoliter and 1 picoliter in size.

20. The system of claim 14, wherein said agarose beads comprise a chemical moiety for imbolization onto a solid surface.

21. The system of claim 19, wherein said chemical moiety is biotin.

22. The system of claim 19, wherein said chemical moiety is an antibody.
